(19) United States
(12) Patent Application Publication
(10) Pub. No.: US 2024/0374207 A1
(43) Pub. Date: Nov. 14, 2024

(54) METHODS AND SYSTEMS FOR DETECTING AND MONITORING HIGH-RESOLUTION ELECTROCORTICOGRAPHIC FEATURES OF NEUROLOGICAL STATES

(71) Applicant: PRECISION NEUROSCIENCE CORPORATION, New York, NY (US)

(72) Inventors: Benjamin I. RAPOPORT, New York, NY (US); Craig H. MERMEL, New York, NY (US); Mark HETTICK, New York, NY (US); Elton HO, New York, NY (US); Demetrios PAPAGEORGIOU, New York, NY (US); Adam J. POOLE, New York, NY (US); Manuel MONGE, New York, NY (US); Kazutaka TAKAHASHI, New York, NY (US); Daniel TRIETSCH, New York, NY (US); Kyle REED, New York, NY (US); Mark MURPHY, New York, NY (US)

(73) Assignee: PRECISION NEUROSCIENCE CORPORATION, New York, NY (US)

(21) Appl. No.: 18/658,871

(22) Filed: May 8, 2024

Related U.S. Application Data
(60) Provisional application No. 63/464,788, filed on May 8, 2023.

(51) Int. Cl. A61B 5/00; A61B 5/293; A61B 5/31; A61B 5/374; A61B 5/384; A61N 1/05; A61N 1/36

(52) U.S. Cl. CPC A61B 5/4836; A61B 5/293; A61B 5/31; A61B 5/374; A61B 5/384; A61B 5/6847; A61N 1/0529; A61N 1/36135; A61B 2560/045; A61B 2560/0468; A61B 2562/046; A61B 2562/164

(57) ABSTRACT

A system for detecting a neurological state of a brain of a patient is disclosed. The system comprises one or more recording arrays, a database, and a processor. The recording arrays comprise a plurality of recording electrodes having a diameter of less than about 1 mm and spaced by less than about 1 mm, and are configured to be minimally invasively inserted to the brain to record electrical signals from a target recording site. The database stores electrophysiological data relating to known electrophysiological signatures associated with neurological states. The processor is configured to receive recorded signals from the recording arrays, access the electrophysiological data from the database, identify electrophysiological signatures in the recorded signals based on the electrophysiological data, and determine the neurological state of the brain based on the identified electrophysiological signatures.

### CROSS-REFERENCE TO RELATED APPLICATIONS

The present disclosure claims priority to U.S. Provisional Patent Application No. 63/464,788 entitled METHODS AND SYSTEMS FOR DETECTING AND MONITORING HIGH-RESOLUTION ELECTROCORTICOGRAPHIC FEATURES OF NEUROLOGICAL STATES filed on May 8, 2023, the contents of which are hereby incorporated by reference in its entirety.

### TECHNICAL FIELD

The present disclosure relates generally to methods, systems, and apparatuses related to identifying a neurological state in a brain of a subject. More particularly, the present disclosure relates to a high-bandwidth system for identifying electrophysiological signatures (i.e., electrocorticographic features in recorded brain signals) indicative of particularly neurological states. The disclosed techniques may be applied to, for example, treating neurological disorders such as epilepsy, Parkinson's disease and other movement disorders, depression, stroke, neurodegenerative disease, and the like. The techniques may also be used to detect pharmacologically induced states (the influence of certain drugs and other chemical agents on brain activity), and to detect certain intentions (such as the intention of a paralyzed person to move).

### BACKGROUND

Electrophysiologic states are understood to change over time in a manner that relates to clinically significant and behavioral events. For example, brain activity may be indicative of epilepsy, depression, or chronic pain. Detecting electrophysiological activity may provide warning signs that provide a window for intervention before crisis occurs.

It has been observed in literature that brain signals may include identifiable features that are indicative of particular conditions. However, such features have generally been observed using conventional, low resolution recording devices that do not have a spatial and/or temporal resolution capable of resolving features of certain conditions at the requisite level of specificity and detail. For example, currently available systems provide very coarse estimates of the electrophysiologic state of a subject. Indeed, neurons in mammals may be as small as tens of microns in diameter and clinically relevant brain states may be represented by the coordinated activity of hundreds or thousands of neurons. As such, electrophysiological activity may span spatial scales from fractions of a millimeter to a few millimeters. On the other hand, conventional systems typically utilize electrode arrays including a small number of large electrodes that provide low spatial resolution (see, for example, FIG. 4), which limits the systems' ability to accurately detect many electrophysiologic states and/or differentiate among similarly presenting electrophysiologic states.

Furthermore, conventional recording electrodes require significant surgery for placement on or within the brain tissue. The degree of invasiveness and collateral damage to normal brain tissue involved with conventional systems limits the population for which such evaluation may be a practical option. Furthermore, where applied, the degree of invasiveness and collateral damage is ultimately a limiting factor on the number of recording electrodes that may feasibly be applied to a subject and, consequently, the amount of data that may be collected for assessment of electrophysiological state. Still further, the magnitude of the procedure results in a limited ability to adjust the spatial placement of the electrodes after initial placement.

Accordingly, it would be advantageous to have a system for identifying a neurological state in a brain of a subject using high-bandwidth neural electrode arrays in order capture the dynamic electrophysiologic state of the brain surface from one or more locations with high spatial resolution, thereby enabling improved diagnosis and monitoring of the neurological state.

### SUMMARY

In one embodiment, the presented disclosure is directed to a system for detecting a neurological state of a brain of a patient, the system comprising: one or more recording arrays configured to be minimally invasively inserted to a target recording site of the brain, each recording array comprising a plurality of recording electrodes having a spacing of less than about 1 mm therebetween, each recording electrode having a diameter of less than about 1 mm and configured to record electrical signals from the target recording site; a database storing electrophysiological data therein, the electrophysiological data relating to one or more known electrophysiological signatures associated with a plurality of potential neurological states; a processor in electrical communication with each of the one or more recording arrays and the database; and a non-transitory, computer-readable medium storing instructions that, when executed, cause the processor to: receive, via the one or more recording arrays, one or more recorded signals from the target recording site, access the electrophysiological data from the database, identify, based on the electrophysiological data, one or more electrophysiological signatures in the one or more recorded signals from amongst the one or more known electrophysiological signatures, and determine, based on the one or more identified electrophysiological signatures, the neurological state of the brain from amongst the plurality of potential neurological states.

In one embodiment, the electrophysiological signatures comprise a collection of time-varying signals collected simultaneously from a collection of electrodes arranged with a particular spatial arrangement across a two-dimensional array.

In one embodiment, the present disclosure is directed to a computer-implemented method for detecting a neurological state of a brain of a patient, the method comprising: receiving one or more recorded signals from a target recording site via one or more recording arrays positioned at the target recording site; accessing electrophysiological data from a database, the electrophysiological data relating to one or more known electrophysiological signatures associated with a plurality of potential neurological states; identifying, based on the electrophysiological data, one or more electrophysiological signatures in the one or more recorded signals from amongst the one or more known electrophysiological signatures; and determining, based on the one or more identified electrophysiological signatures, the neurological state of the brain from amongst the plurality of potential neurological states.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and form a part of the specification, illustrate the embodiments of the invention and together with the written description serve to explain the principles, characteristics, and features of the invention. Various aspects of at least one example are discussed below with reference to the accompanying drawings, which are not intended to be drawn to scale. In the drawings:

FIG. 1 depicts a diagram of an illustrative neural interface system communicatively coupled to an external device in accordance with an embodiment.

FIG. 2 depicts a neural device implanted within a subject at a location between the brain and the dura in accordance with an embodiment.

FIG. 3 depicts a detailed view of an electrode array of a neural device in accordance with an embodiment.

FIG. 4 depicts a conventional neuromodulation system 400 in accordance with an embodiment.

FIG. 5 depicts an illustrative system for detecting neurological states in accordance with an embodiment.

FIG. 6 depicts a comparison of the recording array of FIG. 5 to a conventional electrode array in accordance with an embodiment.

FIG. 7 depicts an array of somatosensory evoked potential (SSEP) signals in accordance with an embodiment.

FIG. 8 depicts a set of example traces in accordance with an embodiment.

FIG. 9 depicts a graphical representation of a correlation map of recorded cortical surface brain signals in accordance with an embodiment.

FIG. 10A depicts a frame of a graphical representation of the cortical surface voltage in accordance with an embodiment.

FIG. 10B depicts a frame of a graphical representation of the cortical surface voltage in accordance with an embodiment.

FIG. 10C depicts a frame of a graphical representation of the cortical surface voltage in accordance with an embodiment.

FIG. 11 depicts a graphical representation of a vector analysis of the recorded brain signals in accordance with an embodiment.

FIG. 12A depicts a frame of a vector analysis of a brain signal in accordance with an embodiment.

FIG. 12B depicts a frame of a vector analysis of a brain signal in accordance with an embodiment.

FIG. 12C depicts a frame of a vector analysis of a brain signal in accordance with an embodiment.

FIG. 13 depicts an example graphical representation of spectral density related to a brain signal response to an administered composition in accordance with an embodiment.

FIG. 14 depicts a flow diagram of an illustrative computer-implemented method for detecting a neurological state of a brain of a patient in accordance with an embodiment.

FIG. 15 illustrates a block diagram of an exemplary data processing system in which embodiments are implemented.

### DETAILED DESCRIPTION

In this disclosure, improved systems and methods are described for identifying electrophysiological signatures in recorded brain activity and determining neurological states of a patient. The expected improvements in performance may be based on higher bandwidth electrophysiologic data that is able to be collected, the ability of the system to practicably interface with diverse and non-adjacent regions of the brain surface, the minimally invasive and reversible nature electrode placement, and the data-handling and computational capacity of the neural recording system.

### Minimally Invasive Neural Interface Systems

The minimally invasive electrode arrays utilized by the systems herein are first described in greater detail. Conventional neural devices typically include electrode arrays that penetrate a subject's brain in order to sense and/or stimulate the brain. However, the present disclosure is directed to the use of non-penetrating neural devices, i.e., neural devices having electrode arrays that do not penetrate the cortical surface. Such non-penetrating neural devices are minimally invasive and minimize the amount of impact on the subject's cortical tissue. Neural interface systems can sense and record brain activity, receive instructions for stimulating the subject's brain, and otherwise interact with a subject's brain as generally described herein. In contrast to conventional systems that require significant surgery for insertion and may require penetration of the cortical surface, the neural interface systems described herein may utilize minimally invasive electrode arrays adapted for insertion with less damage to the body than open surgery and less impact on the subject's cortical tissue.

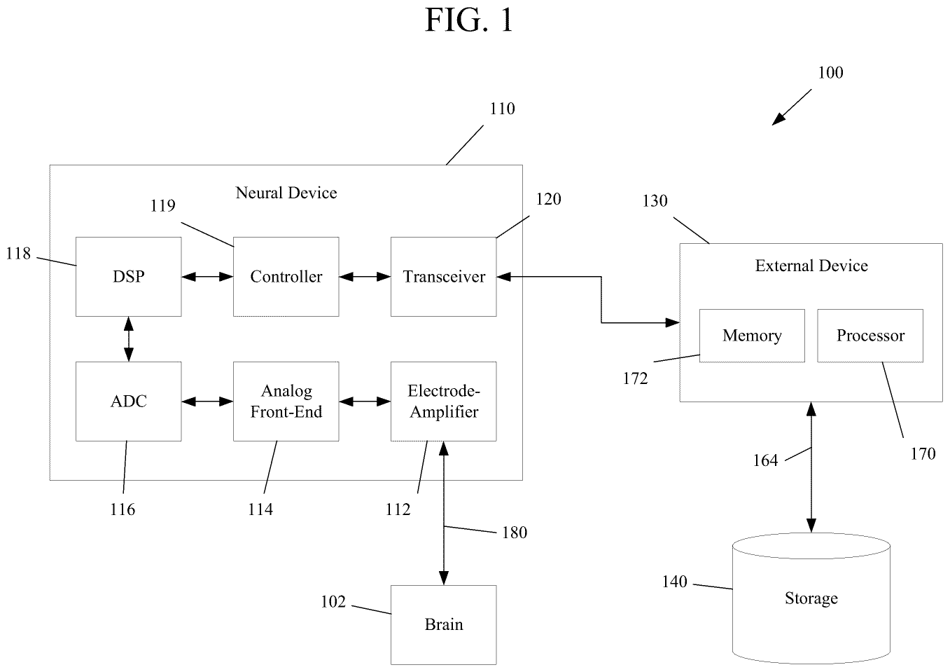

Referring now to FIG. 1, there is shown a diagram of an illustrative system 100 including a neural device 110 that is communicatively coupled to an external device 130. The external device 130 can include any device to which the neural device 110 can be communicatively coupled, such as a computer system or mobile device (e.g., a tablet, a smartphone, a laptop, a desktop, a secure server, a smartwatch, a head-mounted virtual reality device, a head-mounted augmented reality device, or a smart inductive charger device). The external device 130 can include a processor 140 and a memory 142. In some embodiments, the computer system or mobile device can include a server or a cloud-based computing system. In some embodiments, the external device 130 can further include or be communicatively coupled to storage 140. In one embodiment, the storage 140 can include a database stored on the external device 130. In another embodiment, the storage 140 can include a cloud computing system (e.g., Amazon Web Services or Azure). The external device 130 can include a processor 170 and a memory 172. In some embodiments, the external device 130 can include a server or a cloud-based computing system. In some embodiments, the external device 130 can further include or be communicatively coupled to storage 140. In one embodiment, the storage 140 can include a database stored on the external device 130. In another embodiment, the storage 140 can include a cloud computing system (e.g., Amazon Web Services or Azure).

In some embodiments, the electrode array 180 of the neural device 110 can have electrodes that are sufficiently small and spaced at sufficiently small distances in order to define a high-density electrode array 180 that can, accordingly, capture high resolution electrocortical data. Such high-resolution data can be used to resolve electrographic features that can otherwise not be identified using lower resolution electrode arrays. In some embodiments, the electrodes of the electrode array 180 can be from about 10 μm to about 500 μm in width. In one illustrative embodiment, the electrodes of the electrode array 180 can be about 50 μm in width. In some embodiments, the electrodes of the electrode array 180 can be spaced by about 200 μm (i.e., 0.2 mm) to about 3,000 μm (i.e., 3 mm). In illustrative one embodiment, adjacent electrodes of the electrode array 180 can be spaced by about 400 μm.

The neural device 110 can further include a flexible substrate 212 supporting the electrode array 180 and/or other components of the neural device 110. In some embodiments, the flexible substrate 212 can be flexible enough to permit the electrode array 180 to be inserted through an osteotomy into the subdural space 204, then along the cortical surface.

The neural device 110 can include a range of electrical or electronic components. In the illustrated embodiment, the neural device 110 includes an electrode-amplifier stage 112, an analog front-end stage 114, an analog-to-digital converter (ADC) stage 116, a digital signal processing (DSP) stage 118, and a transceiver stage 120 that are communicatively coupled together. The electrode-amplifier stage 112 can include an electrode array, such as is described below, that is able to physically interface with the brain 102 of the subject in order to sense brain signals and/or apply electrical signals thereto. The analog front-end stage 114 can be configured, amplify signals that are sensed from or applied to the brain 102, perform conditioning of the sensed or applied analog signals, perform analog filtering, and so on. The front-end stage 114 can include, for example, one or more application-specific integrated circuits (ASICs) or other electronics. The ADC stage 116 can be configured to convert received analog signals to digital signals. The DSP stage 118 can be configured to perform various DSP techniques, including multiplexing of digital signals received via the electrode-amplifier stage 112 and/or from the external device 130. For example, the DSP stage 118 can be configured to convert instructions from the external device 130 to a corresponding digital signal. The transceiver stage 120 can be configured to transfer data from the neural device 110 to the external device 130 located outside of the body of the subject 102.

In various embodiments, the stages of the neural device 110 can provide unidirectional or bidirectional communications. As indicated in FIG. 1, the external device 130 and the stages 112, 114, 116, 118, 120 of the neural device 110 may be electrically coupled by connectors 154, 156, 158, 160, 162, which may be electrical wires, busses, or any type of electrical connector that enables unidirectional or bidirectional communications as would be known to a person having an ordinary level of skill in the art. Furthermore, the electrode-amplifier stage 112 and the brain 102 of the subject may be electrically coupled by a connector 152, e.g., an electrode array as further described herein. In some embodiments, the connector 152 may be considered part of the electrode-amplifier stage 112. It should be understood that the depicted architecture for the system 100 is merely illustrative and the system 100 can be arranged in various different manners, i.e., stages or other components of the system 100 may be connected differently and/or the system 100 may include additional or alternate stages or components. For example, any of the stages may be arranged and operate in a serial or parallel fashion with other stages of the system 100.

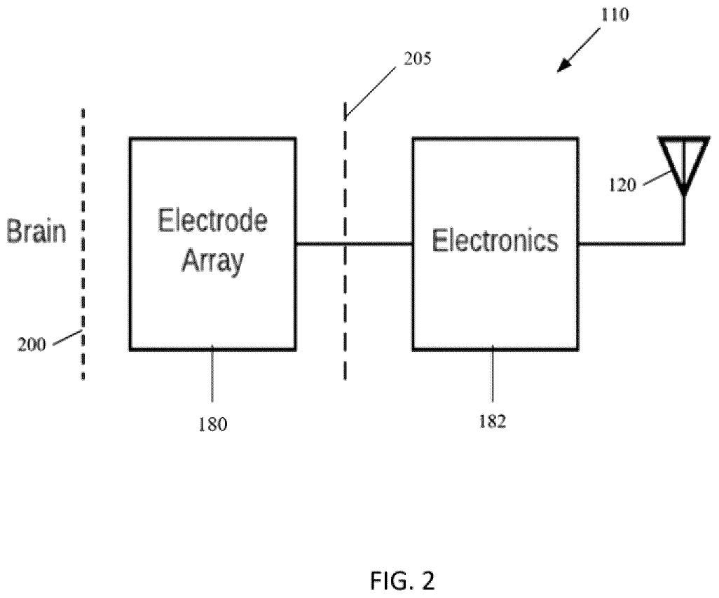

In some embodiments, the neural device 110 described above can include a brain implant, such as is shown in FIG. 2. The neural device 110 may be a biomedical device configured to study, investigate, diagnose, treat, and/or augment brain activity. In some embodiments, the neural device 110 may be positioned between the brain 200 and the dura 205. The neural device 110 can include an electrode array 180 (which may be a component of or coupled to the electrode-amplifier stage 112 described above) that is configured to record and/or stimulate an area of the brain 200. The electrode array 180 can be connected to an electronics hub 182 (which can include one or more of the electrode-amplifier stage 112, analog front-end stage 114, ADC stage 116, and DSP stage 118) that is configured to transmit via wireless or wired transceiver 120 to the external device 130 (in some cases, referred to as a “receiver”).

The electrode array 180 can include non-penetrating cortical surface microelectrodes (i.e., the electrode array 180 does not penetrate the brain 200). Accordingly, the neural device 110 can provide a high spatialresolution, with minimal invasiveness and improved signal quality. The minimal invasiveness of the electrode array 180 is beneficial because it allows the neural device 110 to be used with larger population of patients than conventional brain implants, thereby expanding the application of the neural device 110 and allowing more individuals to benefit from brain-computer interface technologies. Furthermore, the surgical procedures for implanting the neural devices 110 are minimally invasive, reversible, and avoid damaging neural tissue. In some embodiments, the electrode array 180 can be a high-density microelectrode array that provides smaller features and improved spatial resolution relative to conventional neural implants.

In some embodiments, the neural device 110 includes an electrode array configured to stimulate or record from neural tissue adjacent to the electrode array, and an integrated circuit in electrical communication with the electrode array, the integrated circuit having an analog-to-digital converter (ADC) producing digitized electrical signal output. In some embodiments, the ADC or other electronic components of the neural device 110 can include an encryption module, such as is described below. The neural device 110 can also include a wireless transmitter (e.g., the transceiver 120) communicatively coupled to the integrated circuit or the encryption module and an external device 130. The neural device 110 can also include, for example, control logic for operating the integrated circuit or electrode array 180, memory for storing recordings from the electrode array, and a power management unit for providing power to the integrated circuit or electrode array 180.

In some embodiments, the neural device 110 further includes one or more of control logic for operating the stages 112, 114, 116, 118, 120 of the neural device 110 and/or the electrode array 180, a memory for storing recordings from the electrode array and/or instructions for the electrode array, and a power management unit for providing power to various components of the neural device 110 such as the electrode array 180.

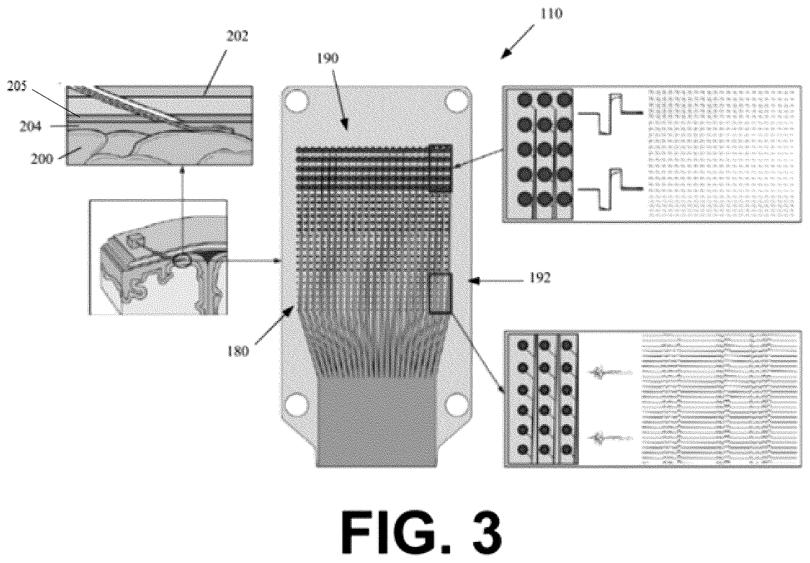

Referring now to FIG. 3, there is shown a diagram of an illustrative embodiment of a neural device 110. In this embodiment, the neural device 110 comprises an electrode array 180 comprising nonpenetrating microelectrodes. The microelectrodes of the electrode array 180 may be arranged in a variety of different configurations and may vary in size. In this particular example, the electrode array 180 includes a first group 190 of electrodes (e.g., 200 μm microelectrodes) and a second group 192 of electrodes (e.g., 20 μm microelectrodes).

The electrode array 180 may be configured for minimally invasive subdural implantation, i.e., insertion into the subdural space 204 between the dura 205 and the brain surface 200 of the subject. As shown in FIG. 3, the electrode array 180 may be delivered using a cranial micro-slit technique. For example, a surgeon may cut an angled slit (e.g., a micro-slit typically having a length of about 400 microns or less, but potentially up to 3 mm in width) through the cranium, followed by an incision in the dura matter, to provide access to the subdural space. Thereafter, the electrode array 180 may be inserted through the micro-slit and into the subdural space 204 in contact with the brain 200. Accordingly, the neural device 110 may provide high spatial-resolution with minimal invasiveness and improved signal quality.

Further, example stimulation waveforms in connection with the first group 190 of electrodes and the resulting post-stimulus activity recorded over the entire array is depicted for illustrative purposes. Still further, example traces from recorded neural activity recorded by the second group 192 of electrodes are likewise illustrated. In this example, the electrode array 180 provides multichannel data that can be used in a variety of electrophysiologic paradigms to perform neural recording of both spontaneous and stimulus-evoked neural activity as well as decoding and focal stimulation of neural activity across a variety of functional brain regions.

In some embodiments, the neural device 110 may be fully implanted in the body. For example, the electrode array 180 may be connected to a self-contained, fully implantable unit that contains the remainder of the components of the neural device 110. In some embodiments, the neural device may include custom hardware and/or an application-specific integrated circuit (ASIC). The unit may have extensive data processing capability, as in the cases of pacemakers and deep brain stimulators, to enable targeted stimulation. In another embodiment, the electrode array 180 may be connected by wires to a unit implanted elsewhere in the body (e.g., the chest wall). In another embodiment the unit may be externally wearable or mounted on the body of the subject (e.g., on the scalp). In some embodiments, all of the electronics may be contained on the electrode array itself, with no wired connection to a secondary unit.

Additional information regarding brain-computer interfaces described herein can be found in Ho et al, The Layer 7 Cortical Interface: A Scalable and Minimally Invasive Brain Computer Interface Platform, bioRxiv 2022.01.02.474656, available https://doi.org/10.1101/2022.01.02.474656, which is hereby incorporated by reference in its entirety.

### Systems for Detecting High-Resolution Electrocorticographic Features of Neurological States

As discussed herein, detection of electrical activity of the brain may be decoded to provide estimates of “brain state.” Detecting neurological states entails sensing neural signals from the brain and identifying a state of the brain based on sensed events or circumstances, e.g., a state related to motor or sensory activity. However, a need exists to expand the scope of detectable neurological states including but not limited to disease states and neural response to treatment, thereby enabling more personalized and context-aware therapy with greater overall efficacy and fewer side effects.

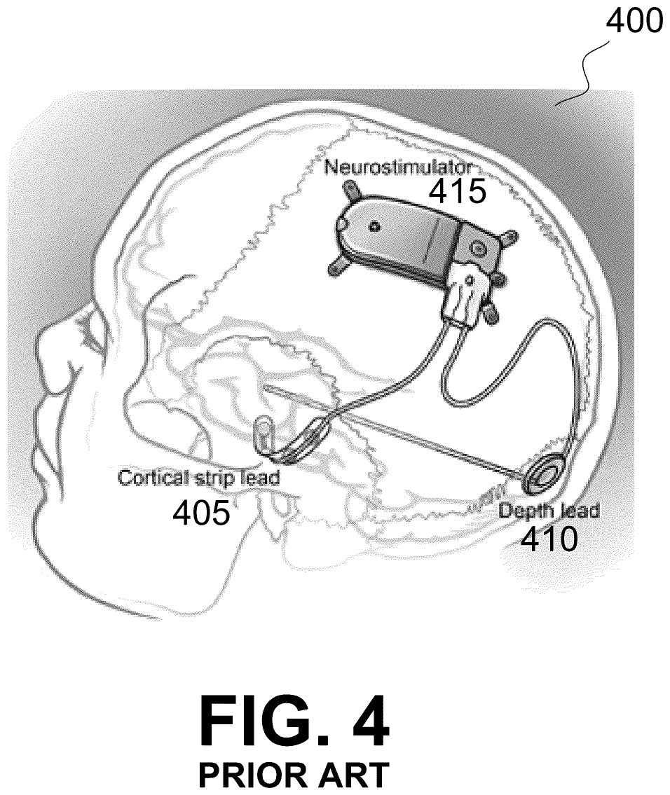

Clinically relevant brain states are represented by the activity of neurons spanning spatial scales from fractions of a millimeter to a few millimeters. However, current neuromodulation systems are limited to low-bandwidth recording arrays including small numbers of large electrodes with low spatial resolution. For example, FIG. 4 depicts a conventional neuromodulation system 400 in accordance with an embodiment. The system 400 may include a cortical strip lead 405 including recording electrodes at a recording site, a depth lead 410 positioned at a stimulation site, and a neurostimulator unit 415 configured to electrically communicate with the cortical strip lead 405 and the depth lead 410. Notably, the cortical strip lead 405 may include a small number of large electrodes (e.g., four electrodes having a diameter of 8 mm each), resulting in coarse estimates of the electrophysiologic state of the brain. The number of electrodes that may be placed is limited by the invasive procedures generally associated with electrode implantation. As such, it would be advantageous to have a system for detecting high-resolution electrocorticographic features using high-bandwidth neural electrode arrays in order capture and identify the dynamic electrophysiologic state of the brain surface in high spatial and temporal resolution.

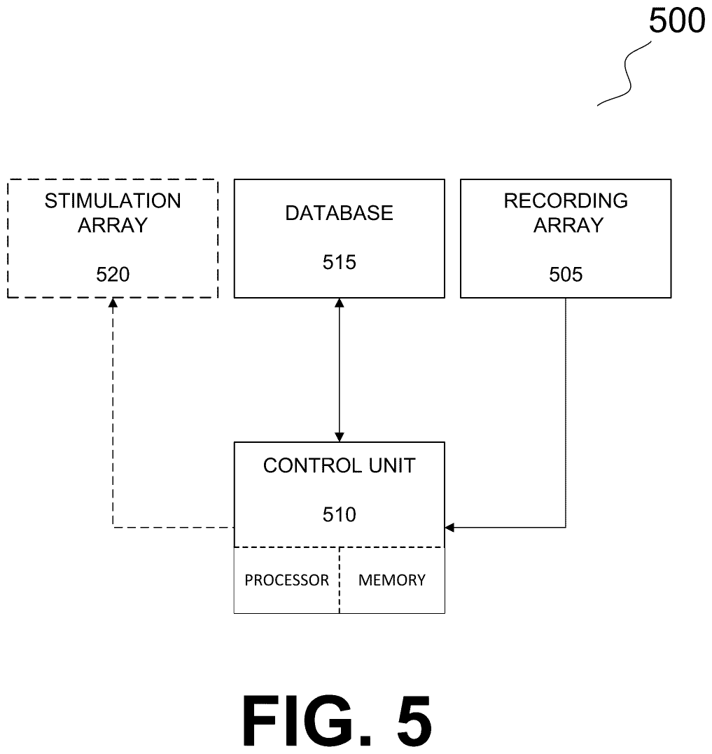

Turning now to FIG. 5, a block diagram of an illustrative system for detecting neurological states is depicted in accordance with an embodiment. The system 500 includes one or more recording arrays 505, a control unit 510 including a processor and a memory, and a database 515. The one or more recording arrays 505 and the database 515 are each in electrical communication with the control unit 510. In some embodiments, the system 500 may further include one or more stimulation arrays 520.

Each of the one or more recording arrays 505 may include a plurality of recording electrodes capable of recording electrical signals from a target recording site. The recording array 505 may be configured to be minimally invasively inserted to the target recording site of the brain as described herein. For example, the recording array 505 may be delivered to the subdural space and/or additional locations using a cranial micro-slit technique as described herein with respect to the electrode array 180. It should be understood that each recording array 505 may be a thin-film, two-dimensional electrode array such as the electrode array 180 as described herein with respect to FIGS. 2-3 and may include any of the features and/or functions as described with respect to the electrode array 180.

In some embodiments, each recording array 505 may be a high-density array that enables greater spatial resolution. For example, the recording electrodes may be arranged with a spatial density of about 100 electrodes/cm², about 200 electrodes/cm², about 300 electrodes/cm², about 400 electrodes/cm², about 500 electrodes/cm², about 600 electrodes/cm², about 700 electrodes/cm², more than 700 electrodes/cm², or individual values or ranges therebetween. Accordingly, the recording electrodes may be spaced from one another by about 1 mm, about 500 μm, about 400 μm, about 250 μm, about 100 μm, about 50 μm, less than about 50 μm, or individual values or ranges therebetween.

In some embodiments, the recording array 505 may include about 529 electrodes. However, it is contemplated that the recording array 505 may include about 100 electrodes, about 200 electrodes, about 300 electrodes, about 400 electrodes, about 500 electrodes, about 1000 electrodes, about 1024 electrodes, greater than 1000 electrodes, or individual values or ranges therebetween.

In some embodiments, the recording electrodes of each recording array 505 may be cortical surface microelectrodes that are configured not to penetrate surface of the brain. In some embodiments, each recording electrode may have a diameter of less than about 1 mm. For example, each recording electrode may have a diameter of about 50 μm. However, it is contemplated that the recording electrodes may have a diameter of about 20 μm, about 40 μm, about 60 μm, about 80 μm, about 100 μm, about 200 μm, about 300 μm, about 400 μm, about 500 μm, greater than about 500 μm, or individual values or ranges therebetween. In some embodiments, the recording electrodes may include combinations of the electrode sizes recited herein.

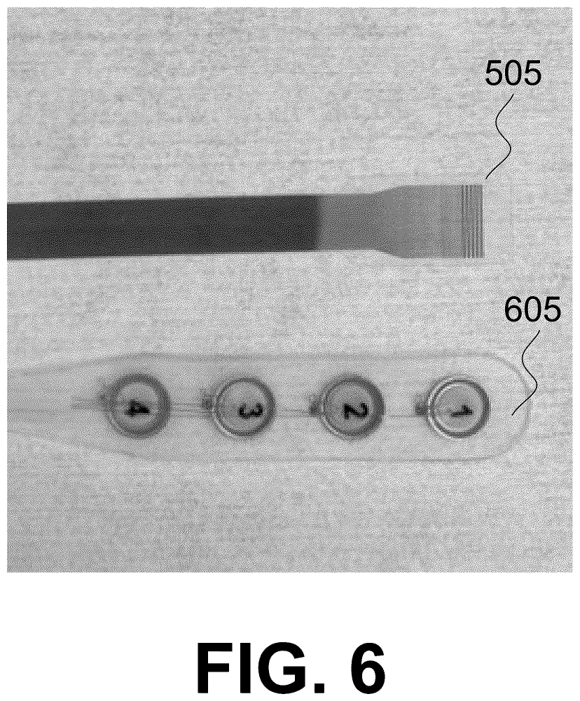

The recording array 505 as described herein may enable collection of higher bandwidth electrophysiologic data. Given that neuron cell bodies may be tens of microns in diameter, the size and arrangement of the recording electrodes enables the recording array 505 to sense signals from the target recording site with a spatial resolution at a similar scale to the neuron cell bodies. Furthermore, the recording array 505 may have a total number of electrodes that is several orders of magnitude greater than conventional recording arrays for sensing neural activity. Referring now to FIG. 6, a comparison of the recording array 505 of FIG. 5 to a conventional electrode array is depicted in accordance with an embodiment. The recording array 505 as shown may include about 500 electrodes of a diameter of about 50 μm arranged in a density of over 100 electrodes/cm². In contrast, a conventional electrode array 605 may include 4 electrodes of a diameter of about 4 mm to about 8 mm spaced linearly by about 1 cm. Accordingly, the recording array 505 may be able to more accurately detect the clinically relevant states of the brain. Still further, the recording array 505 may be able to detect additional clinically relevant states and/or additional details related to the clinically relevant states over conventional recording arrays.

As described, in conventional systems, the invasive nature and the damage to the tissue associated with implantation generally restricts the number of recording arrays and/or the number of locations at which the recording arrays may be positioned. In contrast, the recording arrays 505 may be configured to be minimally invasively inserted as described herein, thus increasing the number of recording arrays that may be feasibly applied to a subject. In some embodiments, the one or more recording arrays 505 may include a plurality of recording arrays 505. In some embodiments, multiple recording arrays 505 may be placed at the same region of the brain. In some embodiments, the recording arrays 505 may be placed at the different regions of the brain. For example, recording arrays 505 may be placed in two or more adjacent regions of the brain. In another example, recording arrays 505 may be placed in two or more non-adjacent regions of the brain. In some embodiments, the recording arrays 505 may be re-positionable by minimally invasive techniques after initial placement in order to improve the data being collected. In some embodiments, the recording arrays 505 may be removable from the subject by minimally invasive techniques after a period of use. Accordingly, the system 500 may be able to collect a greater volume of electrophysiological data and/or a more diverse set of electrophysiological data (i.e., representing a great number of regions of the brain) over conventional systems.

The control unit 510 may be in electrical communication with the one or more recording arrays 505 in order to receive sensed electrophysiological signals therefrom. In some embodiments, the control unit 510 includes a processor and a memory such as a non-transitory, computer-readable medium storing instructions for receiving sensed electrophysiological signals and/or identifying a neurological state of the brain based on the electrophysiological signals. It should be understood that the control unit 510 may include any number of components of the neural device 110 as described herein with respect to FIGS. 1-3 (e.g., the electrode-amplifier stage 112, an analog front-end stage 114, an ADC stage 116, a DSP stage 118, and/or a transceiver stage 120) and may include any of the features and/or functions as described with respect to the neural device 110.

The database 515 may be in electrical communication with the control unit 510 to receive electrophysiological data therefrom and/or to transmit data thereto. The database 515 may store data related to identified electrophysiological signals. For example, neurological states may be associated with particular electrophysiological signals and/or electrophysiological patterns on each electrode in a two-dimensional array that are indicative of the neurological state. Such electrophysiological signals and/or electrophysiological patterns may be referred to as “electrophysiological signatures” and/or “electrophysiological fingerprints” (representative of the two-dimensional nature of the electrode array 180 and how each mappable element can represent a unique identifying characteristic) for the particular neurological state (such terms may be used interchangeably herein).

In some embodiments, the electrophysiological signatures may include a single signal including one or more identifiable characteristics. For example, the signal may be visually depicted as an array or “map” matching the layout of the recording array 505, wherein the signal characteristics of each electrode in the recording array 505 are depicted therein such that features and/or patterns may be identified. In some embodiments, the electrophysiological signatures may include a series or a plurality of signals including one or more identifiable characteristics. For example, the series of signals may be visually depicted as an animation or video of the array (e.g., a moving color map) showing the changes in the signal characteristics over the series at each electrode in the recording array 505. In some embodiments, the identifiable characteristics may be absolute characteristics. In some embodiments, the identifiable characteristics may be relative characteristics, e.g., with respect to other locations within the signal and/or other signals in the series of signals. For example, the identifiable characteristics may be a particular pattern or sequence within the series of signals. In some embodiments, the electrophysiological signatures include one or more characteristics of a single recorded signal, e.g., amplitude, wavelength, frequency, signal width, intensity, anatomic location (i.e., within the brain), and the like. In some embodiments, the electrophysiological signatures may include one or more characteristics of a set or series of recorded signals. For example, the electrophysiological signatures may include a relative characteristic (i.e., changes to a particular characteristic) between the series of recorded signals. In another example, the electrophysiological signatures may include a pattern related to one or more characteristics between the series of recorded signals. However, it should be understood that various additional types of distinct, identifiable features, as would be apparent to a person having an ordinary level of skill in the art, may serve as electrophysiological signatures as described herein.

The database 515 may store information related to the electrophysiological signatures, e.g., sample signals, patterns, characteristics, and the like that may be used by the control unit 510 to detect the signatures within recorded signals, thereby identifying the particular neurological state in the brain. In some embodiments, the database 515 may be stored on the memory of the control unit 510. In some embodiments, the database 515 may be stored at another location accessible to the control unit 510, e.g., a remote computer, server, cloud database, and/or the like.

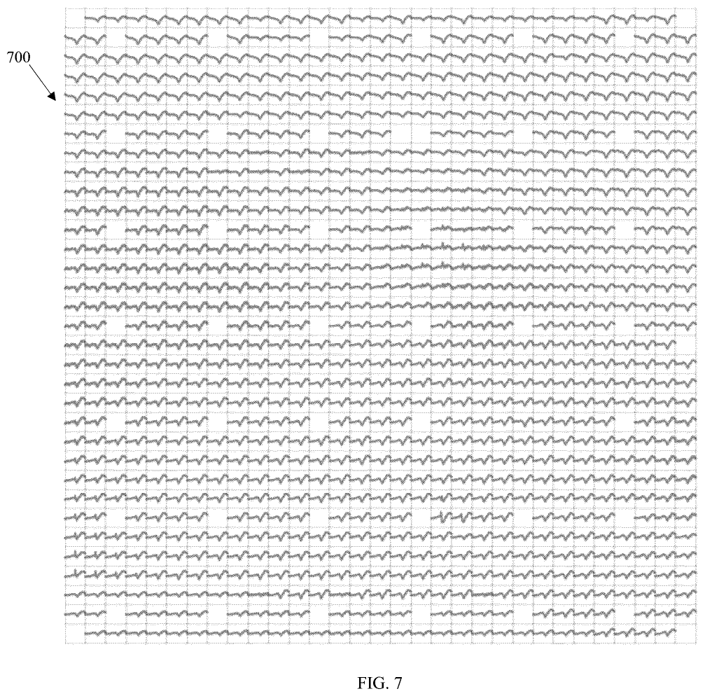

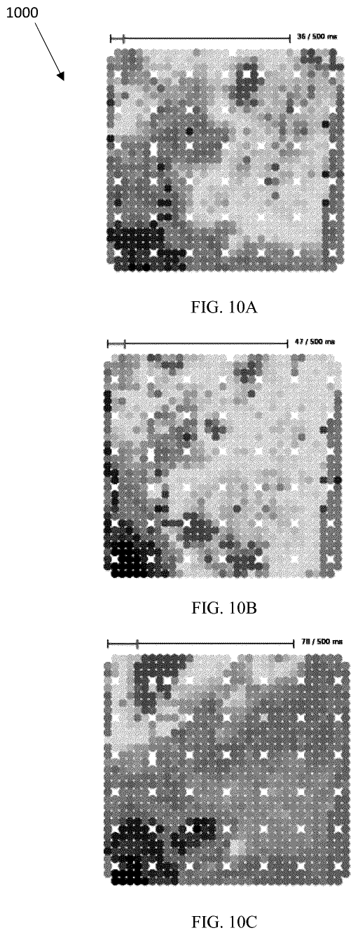

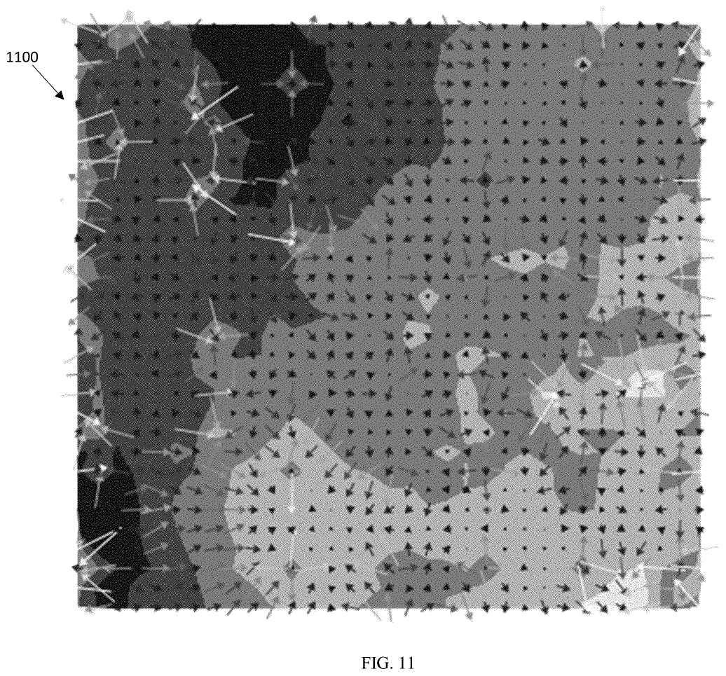

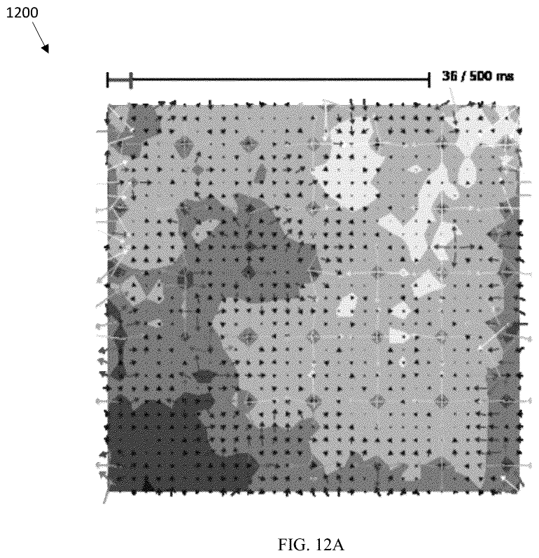

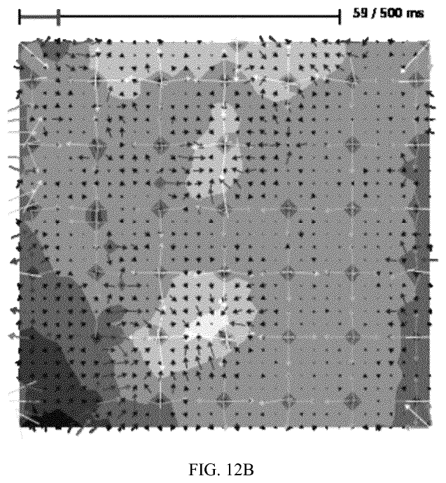

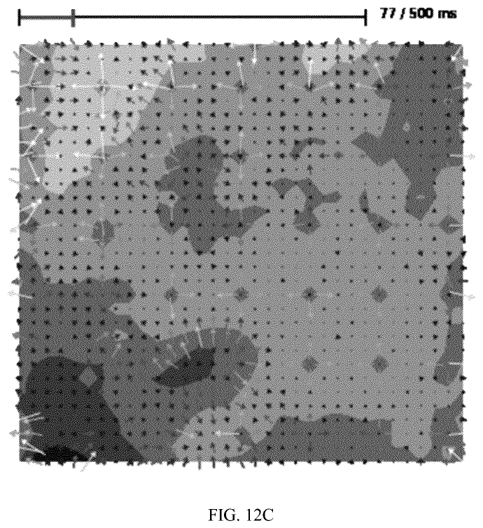

In a first example, the control until 515 may compare the recorded signals to the information in the database in order to identify the neurological state of the brain. In some embodiments, the control unit 510 may access a database 515 to access information related to one or more electrophysiological signatures. The information may be used by the control unit 510 to compare and/or evaluate with respect to recorded signals received from the recording array 505. Accordingly, the control unit 510 may identify an electrophysiological signature within the recorded signals, thereby determining a neurological state of the brain (i.e., the neurological state associated with the electrophysiological signature). Conversely, the control unit 510 may rule out the presence of the electrophysiological signature within the recorded signals, thereby ruling out the particular neurological state associated with the electrophysiological signals. Electrophysiological signatures could be identified from raw electrophysiologic data (e.g., as shown in FIG. 7) or parameters derived from electrophysiologic data, including correlation maps (e.g., as shown in FIGS. 9-10C) or vector analyses (e.g., as shown in FIGS. 11-12C). The association between an electrophysiologic signature and the neurological state may be made in an explicit fashion, or it may be learned, for example using machine learning, in a partially or fully automated manner. In some embodiments, the neurological state may be determined from the electrophysiological signature in either a continuous manner or an iterative manner. The continuous manner may produce a neurological state for each recorded brain signal based on a comparison with the electrophysiological signatures. The iterative manner may produce a neurological state for selected recorded brain signals. The recorded brain signals can be selected at intervals of about 100 ms or less. Both the continuous manner and the iterative manner enable low latency in the determination of the neurological state and permit effectively real-time decoding of a dynamic neural state (as might be the case for decoding a motor intention, a fluctuating state of consciousness, the dynamic level of a drug, or an evolving epileptic seizure).

In a second example, the control unit 510 may transmit signals to the database 515 in order to enable detection of new electrophysiological signatures and identification of new neurological states, and/or to improve detection of known electrophysiological signatures and identification of known neurological states. In some embodiments, the control unit may transmit recorded signals 515 from the recording array 505 to expand or update the information in the database for particular neurological states, e.g., to improve sensing and/or expand sensing to additional symptoms or conditions. For example, the control unit 510 may receive additional patterns or features for recognition in the recorded signals that relate to a particular symptom or disorder. For example, signals may be recorded using a recording array 505 from a patient known to be experiencing a particular neurological state. The recorded signals may then be used by the control unit 510 and/or the database 515 to identify electrophysiological signals for the particular neurological state.

It should be understood that a particular system 500 may be tailored to a particular disorder and/or a set of symptoms associated with the subject, and thus may be programmed and/or re-programmed as such. For example, a particular instance of the system 500 may be configured to detect sub-states and/or symptoms of a particular neurological disorder. Accordingly, the system 500 may be used for monitoring of a patient with a known neurological disorder over time in order to provide treatment based on a sub-state thereof at particular times.

In some embodiments, the system 500 further can include at least one stimulation array 520. The stimulation array 520 may include one or more stimulation electrodes capable of stimulating the brain with electrical signals delivered to a target stimulation site. The stimulation array 520 may be configured to be surgically inserted to the target stimulation site of the brain by conventional techniques. In some embodiments, the target stimulation site may be within the deep brain tissue. In some embodiments, the stimulation array 520 can include a single depth electrode. In some embodiments, the stimulation array 520 can include a plurality of depth electrodes. In some embodiments, the at least one stimulation array 520 can be a single stimulation array 520. In some embodiments, the at least one stimulation array 520 can be a plurality of stimulation arrays 520. In some embodiments, multiple stimulation arrays 520 may be placed at the same region of the deep brain and/or at different regions of the deep brain. It should be understood that the stimulation array 520 of the system 500 may include a conventional depth lead having features and/or functions as would be understood by a person having an ordinary level of skill in the art. In some embodiments, the stimulation array 520 can include a surface electrode array configured for stimulation at the cortical surface, similarly to the electrode arrays 180 described above. In some instances, the stimulation array 520 may have any of the features of the recording arrays 505. Accordingly, the stimulation array 520 may be controlled by the control unit 510 to administer stimulation to the brain tissue based on an identified neurological state as described herein.

As generally described above, electrophysiologic signatures can be identified either directly from raw, recorded electrophysiologic data or parameters and/or data derived from the recorded electrophysiologic data. Examples of such derived parameters and/or data are shown in FIGS. 7-12C and described below.

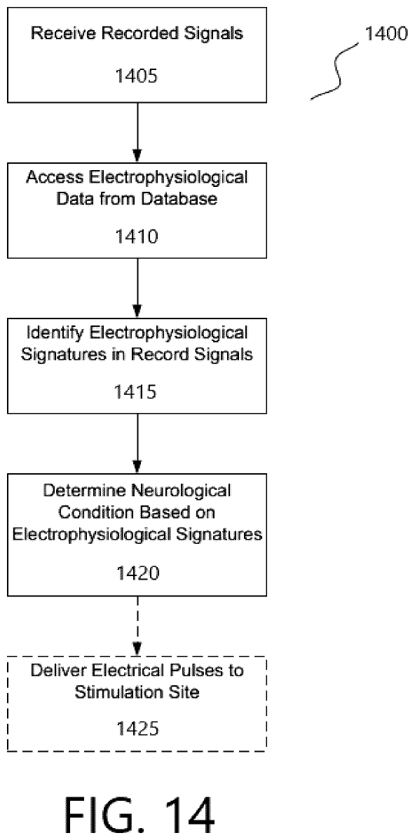

Referring now to FIG. 7, an array 700 of somatosensory evoked potential (SSEP) signals is shown in accordance with an embodiment. Each of the depicted signals is located within the array 700 at the spatial position corresponding to the electrode of the neural interface electrode array 180 from which the signal was recorded. In this particular example, the depicted array 700 corresponds to a 31×33 electrode array 180. The illustrated array 700 of SSEP signals was recorded from a region spanning limb sensory and motor functional areas. The SSEP signals can be processed and compared to electrophysiological signatures to determine a neural state of a patient, as described further in FIG. 14.

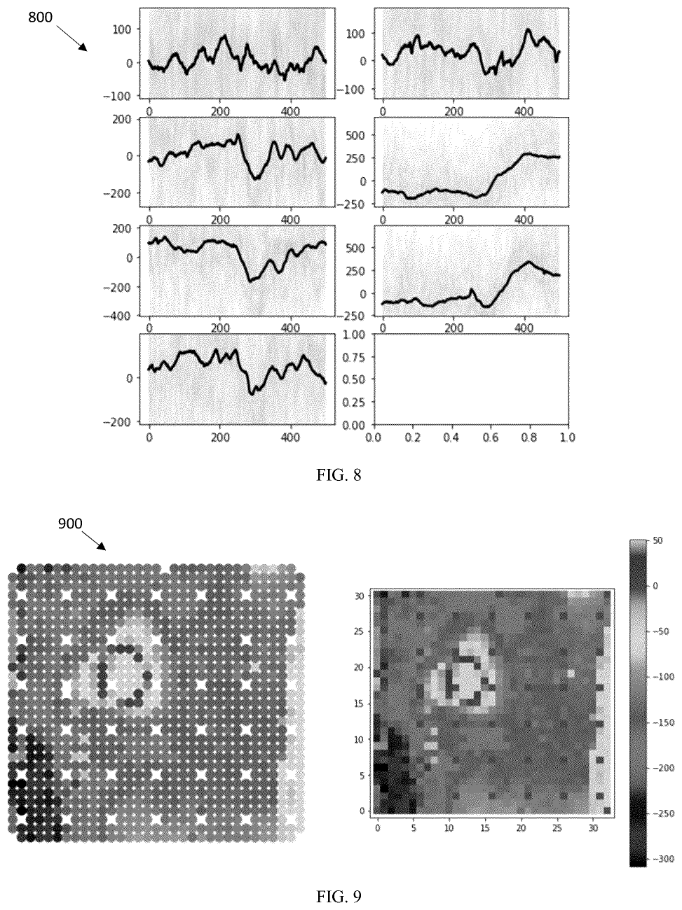

Referring now to FIG. 8, a set of example traces 800 is shown in accordance with an embodiment. The example traces were recorded from a pig that was implanted with a neural device located at the sensorimotor cortex. Each of the illustrative traces represent the signal taken at an individual electrode, indicating that the neural devices 110 described herein can be utilized to resolve highly specific electrophysiologic signals across the cortical surface. Accordingly, the data recorded using the neural devices 100 described herein can be used to identify a variety of different electrophysiological signatures.

Referring now to FIG. 9, a graphical representation 900 of a correlation map of recorded cortical surface brain signals is shown in accordance with an embodiment. A correlation map can be generated from brain signals recorded via an electrode array 180 to identify features and/or patterns from the recorded brain signal data. In this example, the correlation map is illustrating the Pearson correlation coefficient between the time-varying signals on electrodes in a 1024-channel array considered pair-wise. At each electrode site, the correlation coefficient can be computed relative to the reference electrode indicated by a diamond shape. The colors can indicate the correlation coefficient (r²). The correlation coefficient can range from 0 (signified by blue) to 1 (signified by red). The correlation of the recorded brain signals can correspond to certain electrophysiological signatures, which can in turn be used to determine a neural state of the patient.

Referring now to FIGS. 10A-C, frames of a graphical representation 1000 of the recorded brain signals are shown in accordance with an embodiment. FIGS. 10A-C depict frames at successive time points of an SSEP cortical surface voltage map generated from SSEPs recorded via a neural interface electrode array 180. The cortical surface voltage map can by formed by taking the digitized values of an analog-to-digital converter and mapping them to numeric values. As shown, the a first color blue signifies the lowest voltages, and a second color signifies the highest voltages, with a third color green signifying voltages between the aforementioned thresholds.

Referring now to FIG. 11, a graphical representation 1100 of a vector analysis of the recorded brain signals is shown in accordance with an embodiment. The raw cortical surface voltage, as described in FIGS. 10A-C, may be used to form a surface plot. The voltage contours can be analyzed by taking a first spatial derivative at each time point to produce a gradient vector field for each time point. The vector analysis can include determining the magnitude and direction of the rate of change of the signal voltages across the cortical surface as recorded by the electrode array 180. In some embodiments, the magnitude and/or direction of the rate of change in recorded brain signals across the cortical surface can be used to determine a neural state of the patient.

Referring now to FIGS. 12A-C, frames 1200 of a vector analysis across several brain signals is shown in accordance with an embodiment. The vector analysis can identify how the brain signals are changing over time, which can enable identification of a neural state based on an electrophysiological signature. In particular, the vector analysis can identify the magnitude and direction of the change of the voltage contours of the cortical surface, as described in FIG. 11, which can be indicative of different neurological states being experienced by the subject. Accordingly, vector analyses on the data recorded by the neural device 110 can be utilized to derive electrophysiological signatures, which can in turn be utilized to identify neurological states associated with the subject.

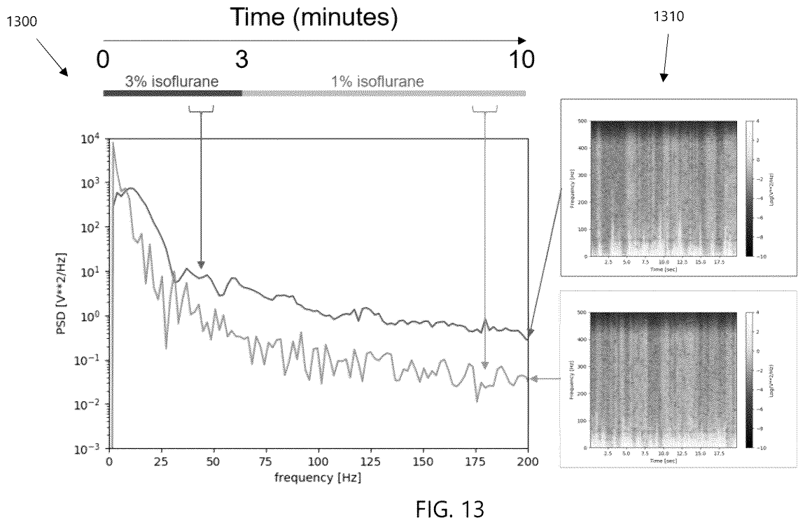

One neurological state that could be identified using the techniques described herein is the response to certain pharmacological or anesthetic agents. This is conveniently illustrated in a case of an anesthetic agent. Referring now to FIG. 13, an example graphical representation 1300 of spectral density on a single electrode in a 1,024-electrode array, related to a brain signal response to an administered composition is shown in accordance with an embodiment. In this example, 3% isoflurane was administered to a subject (a female Gottingen mini-pig) over a three-minute induction period, which was then reduced to 1% isoflurane for maintenance of general anesthesia. During the entire period, a 1,024-electrode array 180 implanted on the right prefrontal cortex of the subject was utilized to record the electrophysiologic response exhibited by the subject. The cortical surface voltage on each electrode can be sampled at 30,000 samples per second. A graphical representation 1310 of the frequency of the separate brain signals in the time and frequency domains is also shown. As can be seen, the spectral density and time-frequency spectrograms of the recorded signals can be analyzed as shown for each of the 1,024 electrodes in the array, generating a rich, high-dimensional data set containing time, frequency, and spatial information about the neural signals from the cortex in the region of the implanted electrode array 180. This data set can provide distinct signatures of the neural state induced by the identity and level of the pharmacologic agent being used (in this case, an anesthetic agent), and these signatures can also be used to identify the agent itself.

Turning now to FIG. 14, a flow diagram of an illustrative computer-implemented method 1400 for detecting a neurological state of a brain of a patient by the system 500 is depicted in accordance with an embodiment. For example, the method 1400 may be carried out by the processor of the control unit 510 upon execution of the instructions stored on the memory. The method 1400 includes receiving 1405 one or more recorded signals (i.e., collected at a target recording site of the brain) from the recording array(s) 505, accessing 1410 electrophysiological data from a database, identifying 1415 one or more electrophysiological signatures in the one or more recorded signals based on the electrophysiological data, and determining 1420 a neurological state of the brain based on the one or more identified electrophysiological signatures. In some embodiments, the method 1400 may further include delivering 1425 a set of electrical pulses to a target stimulation site via the stimulation array(s) 520 based on the determined neurological state of the brain.

As shown in FIG. 3, each recording array 505 may electrically communicate with the control unit 510 to communicate the recorded signals thereto. It should be understood that the control unit 510 may continuously receive 1405 recorded signals from the recording array 505 at a temporal resolution suitable for determining a neurological state of the brain as would be known to a person having ordinary skill in the art. It should be understood that identifiable features of recorded signals have been observed in literature and clinical practice using conventional, low resolution recording devices. Advantageously, as described herein, recording electrophysiological signals at an improved spatial and/or temporal resolution by the disclosed devices (e.g., the recording array 505) enable recognition of electrophysiological signatures at an enhanced level of specificity and detail, thereby facilitating improved levels of diagnostic monitoring and therapeutic precision. Recognition of electrophysiological signatures at an enhanced level of specificity and detail is only possible with a high-resolution array. As discussed in FIG. 6, conventional electrode arrays do not record at an appropriate resolution to enable recognition of different electrophysiological signatures to determine neural states. Accordingly, the control unit 510 may collect recorded signals configured for identifying 1415 features of interest therein, and may continually identify and/or update identified features over time.

The electrophysiological data accessed 1410 from the database may include various types of information. In some embodiments, the database is a database 515 as described herein with respect to FIG. 5 and may include any of the various types of information disclosed with respect to the database 515. In some embodiments, the electrophysiological data may include one or more known electrophysiological signatures. For example, the database may include a library of known electrophysiological signatures. The known electrophysiological signatures may be documented and/or known to be associated with particular neurological states, e.g., through research and/or assessment of recorded signals from historical patients. Accordingly, detecting the known electrophysiological signatures may be indicative of the particular neurological state. In some embodiments, the electrophysiological data may include one or more electrophysiological signatures associated with “suspected” neurological states, e.g., neurological states that the control unit 510 may attempt to detect in the patient. In some embodiments, the electrophysiological data can include the entire library and/or a subset of the library of electrophysiological signatures. For example, the control unit 510 may access 1410 a subset of the library based on known patient parameters (e.g., gender, age, medical history, and the like) such that the control unit 510 may assess neurological states that may be more likely based on the known patient parameters. In some embodiments, the control unit 510 may communicate with the database remotely and access 1410 the electrophysiological data externally from the control unit 510, e.g., at a remote computer, server, cloud database, and/or the like. In some embodiments, the control unit 510 may download and/or store the electrophysiological data on the control unit 510, e.g., on the memory.

In some embodiments, the electrophysiological signatures may include one or more characteristics of a single recorded signal, e.g., amplitude, wavelength, frequency, signal width, intensity, location (e.g., within the brain), and the like. In some embodiments, the electrophysiological signatures can include one or more characteristics of a set or series of recorded signals. For example, the detected features may include a relative characteristic (i.e., changes to a particular characteristic) between the series of recorded signals. In another example, the detected features may include a pattern related to one or more characteristics between the series of recorded signals, or more complex derived properties of the recorded signals. These features may combine properties of the recorded signals that involve spatial and/or temporal aspects of the signals. However, it should be understood that the electrophysiological signatures may include any detectable features and/or characteristics as described herein within respect to the database 515.

The control unit may identify 1415 one or more electrophysiological signatures in the one or more recorded signals in a variety of manners as would be known to a person having an ordinary level of skill in the art. In some embodiments, the control unit 510 may process the recorded signals to detect one or more features in the recorded signals. The control unit 510 can use one or more analytical techniques to process the recorded signals. For example, the one or more analytical techniques can include at least one of time series analysis, spectral density analysis, vector analysis, waveform analysis, linear or nonlinear filtering, neural network analysis, and derivative analysis.

Thereafter, the detected features may be compared to the electrophysiological data, i.e., known electrophysiological signatures, to identify matches. The electrophysiological signatures can include features specific to certain analytical techniques. For example, an electrophysiological signature can include a spectral density representation which can be matched to a recorded brain signal with the same or similar spectral density to determine a neural state. In some embodiments, the identifying 1415 the one or more electrophysiological signatures is performed according to an algorithm. In some embodiments, the identifying 1415 the one or more electrophysiological signatures is performed by a machine learning algorithm trained to identify electrophysiological signatures from the recorded signals. In some embodiments, the electrophysiological data may include such a machine learning algorithm. Accordingly, the control unit 510 may identify 1415 electrophysiological signatures in the recorded signals.

The control unit 510 may determine a neurological state of the brain based on the one or more identified electrophysiological signatures. In some embodiments, the presence of the identified electrophysiological signature indicates the neurological state. For example, the identified electrophysiological signature may be associated with a particular neurological state as described herein. In some embodiments, a plurality of identified electrophysiological signatures may be associated with a particular neurological state. For example, several electrophysiological signatures may each be associated with a particular neurological state such that the presence of any one of the electrophysiological signatures indicates the neurological state. In another example, the combined presence of all of the associated electrophysiological signatures indicates the neurological state. In another example, the combined presence of a plurality of the electrophysiological signatures indicates the neurological state, e.g., the presence of two out of three associated electrophysiological signatures indicates the neurological state. It should be understood that the criteria for determining a particular neurological state, while based on the identified electrophysiological signatures, may vary in the case of each different neurological state due to the natural complexity and/or heterogeneity that exists from patient to patient with respect to neurological states.

As shown in FIG. 3, the control unit 510 may electrically communicate with each stimulation array 520 to operate the electrodes to deliver 1425 the set of electrical pulses. It should be understood that the set of electrical pulses may include one electrical pulse or a plurality of electrical pulses having varying parameters as would be known to a person having an ordinary level of skill in the art. Furthermore, the set of electrical pulses may be delivered 1425 at a temporal resolution suitable for affecting the determined neurological state of the brain and/or symptoms thereof as would be known to a person having ordinary skill in the art. In some embodiments, the electrophysiologic behavior detected following stimulation can assist in identifying the underlying state. In some embodiments, the control unit 510 may access treatment data from the database. The treatment data may include a set of pulse parameters directed to treat the determined neurological state and/or symptoms thereof. In some embodiments, the treatment data may be stored on the memory or another location accessible to the control unit 510.

In some embodiments, the set of electrical pulses may be configured to electrically stimulate the brain to change the neurological state of the brain. For example, the set of electrical pulses may result in reduction or elimination of one or more symptoms associated with a neurological condition. In some embodiments, the set of electrical pulses may be configured to electrically stimulate the brain to maintain a neurological state of the brain. For example, the set of electrical pulses may prevent onset of one or more symptoms associated with a neurological condition and/or reduce occurrence of the one or more symptoms.

The systems and methods described herein are capable of use for identifying “neurological states” that relate to motor and/or sensory functions of the brain. For example, the neurological states may be related to movement, intent to move a body part (e.g., in cases of paralysis and/or amputation), touching and/or sensing objects, and the like. However, it should be understood the systems and methods described herein are advantageously capable of use for determining non-motor and/or non-sensory neurological states.

Furthermore, neurological states may change over time in a manner that can predict clinically significant and behavioral events before such events are externally apparent and/or at the level of conscious awareness for the patient. Detecting electrophysiologic signatures for such events provide warning signs that provide a window for intervention before crisis occurs, thereby enabling more effective treatment.

In some embodiments, the neurological state relates to epilepsy. The system 500 and/or the method 1400 may be used to identify various epileptic states. For example, the neurological state may be an epileptic or pre-epileptic condition, i.e., the system 500 and/or the method 1400 may be used to diagnose epilepsy. Electrophysiological signatures may manifest before an epileptic condition is otherwise apparent by symptoms and the like. Furthermore, some epileptic conditions may be difficult to diagnose by conventional means. Accordingly, the system 500 and/or the method 1400 may enable early and/or improved diagnosis of epileptic conditions as well as detection and/or treatment thereof. In a particular example, brain patterns (i.e., electrophysiological signatures) may foreshadow a seizure. In another example, subclinical seizure activity may not be readily apparent externally by symptoms but may nonetheless be recognizable by electrophysiological signatures. As such, the electrophysiological signatures indicating subclinical seizure activity and/or an imminent seizure may be capable of detection even before symptoms are triggered and/or apparent to the patient or observers. Accordingly, system 500 and/or the method 1400 may enable detection of subclinical seizure activity and/or imminent seizures, and thus prevention of such episodes by timely intervention. In additional embodiments, the detected electrophysiological signatures may also be indicative of causes and/or origin of a seizure within the brain.

In some embodiments, the neurological state relates to Parkinson's disease. The system 500 and/or the method 1400 may be used to identify various states related to Parkinson's disease. In some embodiments, one or more neurological states may be stages of Parkinson's disease, e.g., early, advanced, late stage, etc. Accordingly, the system 500 and/or the method 1400 may be used to diagnose and/or grade Parkinson's disease in a patient. Electrophysiological signatures may manifest before Parkinson's disease is otherwise apparent by symptoms and the like. Furthermore, Parkinson's disease may be difficult to diagnose by conventional means. Accordingly, the system 500 and/or the method 1400 may enable early and/or improved diagnosis of Parkinson's disease as well as detection and/or treatment thereof.

In some embodiments, the neurological state relates to dementia and/or Alzheimer's disease. The system 500 and/or the method 1400 may be used to identify various states related to dementia and/or Alzheimer's disease. In some embodiments, one or more neurological states may be stages of dementia and/or Alzheimer's disease, e.g., early, advanced, late stage, etc., and/or subtypes of dementia and/or Alzheimer's disease. Additionally, Alzheimer's disease can sometimes be difficult to distinguish from other forms of dementia, and electrophysiologic diagnosis, particularly at early stages, could be helpful for prognostication or treatment. Accordingly, the system 500 and/or the method 1400 may be used to diagnose and/or grade dementia and/or Alzheimer's disease in a patient. Electrophysiological signatures may manifest before dementia and/or Alzheimer's disease is otherwise apparent by symptoms and the like. Furthermore, dementia and/or Alzheimer's disease may be difficult to diagnose by conventional means, especially as related to subtypes. Accordingly, the system 500 and/or the method 1400 may enable early and/or improved diagnosis of dementia and/or Alzheimer's disease as well as detection and/or treatment thereof.

In some embodiments, the neurological state relates to depression. The system 500 and/or the method 1400 may be used to identify various states related to depression. In some embodiments, one or more neurological states may be stages of depression, degree of depression, and/or subtypes of depression. Accordingly, the system 500 and/or the method 1400 may be used to diagnose and/or grade depression in a patient. Electrophysiological signatures may manifest before depression is otherwise apparent by symptoms and the like. Furthermore, depression may be difficult to diagnose by conventional means, especially as related to subtypes. Accordingly, the system 500 and/or the method 1400 may enable early and/or improved diagnosis of depression. In a particular example, the neurological state may relate to relapses and/or suicidality associated with depression. In some embodiments, particular brain patterns (i.e., electrophysiological signatures) foreshadow relapse, suicidality, and/or suicidal episodes. In some embodiments, the electrophysiological signatures may be capable of detection even before the stage or degree of depression and/or suicidality are consciously felt by the patient, i.e., while present only at a subconscious level. Accordingly, system 500 and/or the method 1400 may enable detection of relapses and/or suicidality in a patient, and thus prevention of such episodes by timely intervention.

In some embodiments, the neurological state relates to chronic pain. The system 500 and/or the method 1400 may be used to identify various states related to chronic pain. In some embodiments, one or more neurological states may be stages of chronic pain (i.e., particular episodes or exacerbations there) and/or degree of chronic pain. Accordingly, the system 500 and/or the method 1400 may be used to detect, diagnose and/or grade chronic pain in a patient. Electrophysiological signatures may manifest before chronic pain is otherwise apparent by symptoms and the like. Furthermore, chronic pain may be difficult to diagnose by conventional means. Accordingly, the system 500 and/or the method 1400 may enable early and/or improved diagnosis of chronic pain. In some embodiments, particular brain patterns (i.e., electrophysiological signatures) foreshadow oncoming pain (i.e., pain crises). For example, the electrophysiological signatures may be present and capable of detection even before the pain crisis reaches a threshold of consciousness, i.e., while present only at a subconscious level. Accordingly, system 500 and/or the method 1400 may enable detection of pain crises, pain episodes, and/or exacerbations, and thus prevention of such episodes by timely intervention.

While various neurological states are described herein with respect to detection and/or treatment, it should be understood that such neurological states may be monitored over time by repeated assessment for electrophysiological signatures in order to monitor progress of the patient. For example, after diagnosis and/or detection of a neurological state, a treatment or therapy may be administered to the patient to treat the neurological state. Thereafter, the patient may be monitored to observe reduction and/or elimination of the electrophysiological signatures in recorded signals, thereby indicating successful treatment. Additionally, the patient may be monitored to observe persistence of the electrophysiological signatures, thereby indicating inefficacy of the treatment. In some embodiments, the magnitude and/or frequency of the electrophysiological signatures may indicate a degree of change in the neurological state (e.g., improvement or decline) and/or an efficacy of a treatment or therapy, thereby informing continued care.

In some embodiments, the neurological state relates to an administered composition. In some embodiments, the composition may be a drug. The system 500 and/or the method 1400 may be used to monitor an effect of the composition on the brain activity of the patient. Accordingly, the system 500 and/or the method 1400 may be used to monitor an efficacy of the composition, e.g., as a treatment or therapy. In a particular example, the composition may be an anesthetic. The anesthetic may be applied and brain activity may be recorded to identify electrophysiological signatures associated with an effect of the anesthetic. In some embodiments, the degree of effect of the anesthetic may be determined from the electrophysiological signatures. For example, the electrophysiological signatures to identify a degree of consciousness of the patient in order to determine a threshold or level of consciousness. Such information may be informative for determining guidelines and/or dosing for an anesthetic.

Furthermore, various compositions (e.g., a pharmaceutical for treatment of neurological conditions) may be assessed in a similar manner to identify an efficacy of the pharmaceutical, guidelines, and/or dosing information therefor. Advantageously, the detection of electrophysiological signatures enables objective determination of an effect of the pharmaceutical on the brain. In a particular example, the composition may be an antidepressant. The antidepressant may be administered and brain activity may be recorded to identify electrophysiological signatures associated with an effect of the antidepressant. In some embodiments, the degree of effect of the antidepressant may be determined from the electrophysiological signatures. Such information may be informative for determining efficacy of the composition for treatment of particular conditions in various patient groups, as well as guidelines and/or dosing therefor.

While particular examples of compositions for evaluation using electrophysiological signatures are described herein, it should be understood that the system 500 and/or method 1400 may be applied to various compositions, medications, treatments, and/or therapies to evaluate effects thereof. For example, any existing and/or emerging pharmaceutical may be objectively evaluated for effect and efficacy in the manner described herein. Furthermore, effects and/or efficacy of different pharmaceuticals and/or therapies may be compared. For example, two pharmaceuticals can be shown to be similar in their activity on a patient for a particular neurological state if they cause the same effect on electrophysiological signatures therefor. In some embodiments, this evaluation may provide insights into the “pathway” of activity for particular pharmaceuticals and/or therapies. In some embodiments, the evaluation may be effective for identifying interactions, placebo effects, and the like for various pharmaceuticals and/or therapies.

In some embodiments, the system 500 and/or the method 1400 may be used to monitor a prognosis. For example, after an initial diagnosis, the system 500 and/or the method 1400 may be used to perform repeated evaluation to monitor a progression of a neurological condition. In some embodiments, the collected data may be used for further understanding and/or predicting progression of various conditions in future cases.

In some embodiments, the system 500 and/or the method 1400 may be used to monitor treatment compliance. For example, where a patient is diagnosed with a neurological condition and placed on a treatment plan (e.g., a dosing schedule for a medication), the system 500 and/or the method 1400 may be used to perform repeated evaluation to monitor compliance with the treatment plan. In some embodiments, the collected data may be used to identify a level of compliance, e.g. a missed dose and/or an overdose may be identifiable based on monitoring of the electrophysiological signatures associated with the neurological condition and/or administration of the medication.

As described herein, a machine learning algorithm may be utilized to identify and/or associate electrophysiological signatures with particular neurological states. For example, a machine learning algorithm may be trained using a set of training data. In some embodiments, the set of training data includes clinical data from historical patients, e.g., recorded signals from the brain. The training data may be used to train the machine learning algorithm to identify unique electrophysiological signatures. Furthermore, the training data may be used to train the machine learning algorithm to associate identified electrophysiological signatures with particular neurological states. For example, the training data may include outcomes from patient assessments from historical patients such that there is an indication of the accuracy of the assessment, e.g., wherein a neurological state of the patient was determined and/or verified by other means. Accordingly, the machine learning algorithm may become more proficient in identifying electrophysiological signatures and/or associating signatures with neurological states over time. Thus, a trained machine learning algorithm may identify new and useful electrophysiological signatures, thereby improving the ability of the control unit 510 to identify neurological states and/or expanding the variety of neurological states that may be identified based on electrophysiological signatures. Treatment data from the historical patients may similarly be used to train the machine learning algorithm to identify effective treatments for a neurological state, e.g., stimulation parameters.

In some embodiments, the machine learning algorithms may be continuously trained and thus improved. For example, the machine learning algorithm may be trained using a first set of training data, which may be “seed data.” The seed data may include clinical data from historical patients as described herein. The seed data may be of at least a critical volume to enable the machine learning algorithm to satisfactorily identify electrophysiological signatures and/or neurological states. Following the performance of the method 1400, the clinical data of the patient, including demographic information, brain activity information, treatment information and/or outcome information, may be used to further train the machine learning algorithms. For example, where a particular diagnosis is rejected by a user, the machine learning algorithm may obtain an indication of these outcomes and may be trained over time to provide different and/or better predictions or proposals in similar scenarios. In another example, where a particular diagnosis received positive feedback, the machine learning algorithm may obtain an indication of these outcomes and may be trained over time to provide similar predictions or proposals in similar scenarios. In another example, after treatment is applied, the machine learning algorithm may obtain outcome data associated with the success or failure of the treatment and may be trained over time to recognize treatment parameters with a high likelihood of success and/or a low likelihood of success. Accordingly, live cases may be used to form a second set of training data, which may be “refinement data” that is used on a continual basis to re-train the machine learning algorithms.

The devices, systems, and methods as described herein are not intended to be limited in terms of the particular embodiments described, which are intended only as illustrations of various features. Many modifications and variations to the devices, systems, and methods can be made without departing from their spirit and scope, as will be apparent to those skilled in the art.

It should be understood that the devices described herein, e.g., the recording array 505 and/or the entire system 500, provide rigor and structure on collected data. For example, the design of the recording array 505 enables collection of data from a plurality of patients over time in a consistent manner because the recording array 505 has a design with constant parameters (i.e., electrode number, spacing, layout, etc.). Accordingly, the data collected from various patients is highly comparable in a manner beyond the capability of conventional systems. However, it should be understood that additional systems may be utilized to perform techniques similar to those described herein.

While various exemplary conditions, symptoms, diseases, and disorders are described herein, the method 1400 may be applied to various additional conditions, symptoms, diseases, and disorders as would be apparent to a person having an ordinary level of skill in the art. Furthermore, the target recording site(s) and the target stimulation site(s) may be selected based on a target symptom and/or disorder.

In some embodiments, the system 500 may be configured to communicate with additional external devices. For example, the control unit 510 may communicate with an external device in a similar manner as the control unit 510 communicates with the database 515 as described with respect to FIG. 5. In some embodiments, the control unit 510 may communicate with external devices to program the control unit 510, re-program the control unit 510, and/or to receive software updates.

In the present disclosure, identification of electrophysiological signatures may include “electrophysiologic phenotypes” and/or “electrophysiologic biomarkers.” Electrophysiologic phenotypes may refer to electrophysiologic subtypes of a particular disorder (e.g., subtypes of epilepsy or depression) that are relatively constant in the patient and/or are present for longer periods of time. Conversely, electrophysiologic biomarkers may reflect transient changes, even within an individual person, from time to time that may reflect various markers of biological condition and/or health. For example, electrophysiological biomarkers may be associated with a level of consciousness. It should be understood that electrophysiological signatures as discussed herein may include both electrophysiologic phenotypes and electrophysiologic biomarkers.

In some embodiments, the control unit 510 may communicate with external devices to receive information for the database and/or to expand or update the information in the database, e.g., to improve sensing and/or expand sensing to additional symptoms or conditions. For example, the control unit 510 may receive additional patterns or features for recognition in the recorded signals and/or additional pulse patterns to deliver to the deep brain to treat a symptom or disorder. It should be understood that a particular system 500 may be tailored to a particular disorder and/or a set of symptoms associated with the subject, and thus may be programmed and/or re-programmed as such.

### Data Processing Systems for Implementing Embodiments Herein

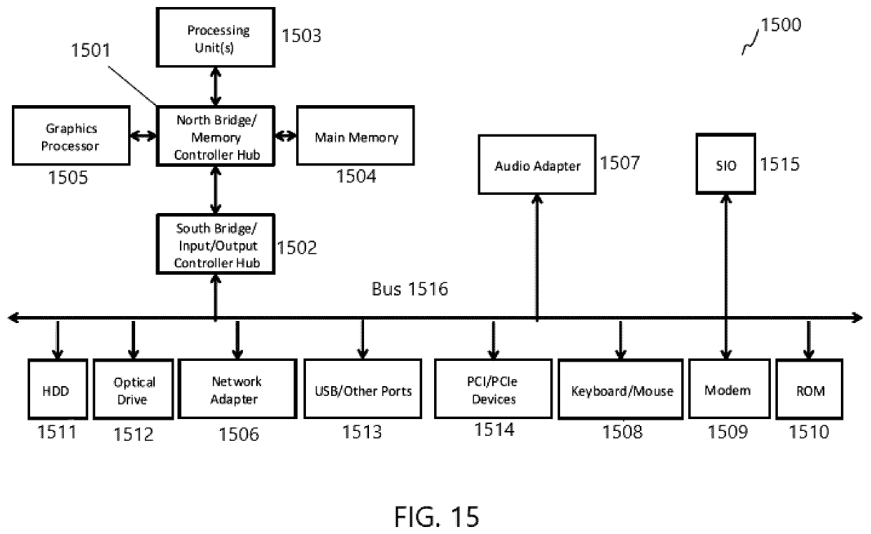

FIG. 15 illustrates a block diagram of an exemplary data processing system 1500 in which embodiments are implemented. The data processing system 1500 is an example of a computer, such as a server or client, in which computer usable code or instructions implementing the process for illustrative embodiments of the present invention are located. In some embodiments, the data processing system 1500 may be a server computing device. For example, data processing system 1500 can be implemented in a server or another similar computing device operably connected to a neural device 110 or a system 500 as described above. The data processing system 1500 can be configured to, for example, transmit and receive information related to a patient, electrophysiological signatures, and/or a treatment plan with the system 500.

In the depicted example, data processing system 1500 can employ a hub architecture including a north bridge and memory controller hub (NB/MCH) 801 and south bridge and input/output (I/O) controller hub (SB/ICH) 802. Processing unit 1503, main memory 1504, and graphics processor 1505 can be connected to the NB/MCH 1501. Graphics processor 1505 can be connected to the NB/MCH 1501 through, for example, an accelerated graphics port (AGP).

In the depicted example, a network adapter 1506 connects to the SB/ICH 1502. An audio adapter 1507, keyboard and mouse adapter 1508, modem 1509, read only memory (ROM) 1510, hard disk drive (HDD) 1511, optical drive (e.g., CD or DVD) 1512, universal serial bus (USB) ports and other communication ports 1513, and PCI/PCIe devices 1514 may connect to the SB/ICH 1502 through bus system 1516. PCI/PCIe devices 1514 may include Ethernet adapters, add-in cards, and PC cards for notebook computers. ROM 1510 may be, for example, a flash basic input/output system (BIOS). The HDD 1511 and optical drive 1512 can use an integrated drive electronics (IDE) or serial advanced technology attachment (SATA) interface. A super I/O (SIO) device 1515 can be connected to the SB/ICH 1502.

An operating system can run on the processing unit 1503. The operating system can coordinate and provide control of various components within the data processing system 1500. As a client, the operating system can be a commercially available operating system. An object-oriented programming system, such as the Java™ programming system, may run in conjunction with the operating system and provide calls to the operating system from the object-oriented programs or applications executing on the data processing system 1500. As a server, the data processing system 1500 can be an IBM® eServer™ System® running the Advanced Interactive Executive operating system or the Linux operating system. The data processing system 1500 can be a symmetric multiprocessor (SMP) system that can include a plurality of processors in the processing unit 1503. Alternatively, a single processor system may be employed.

Instructions for the operating system, the object-oriented programming system, and applications or programs are located on storage devices, such as the HDD 1511, and are loaded into the main memory 1504 for execution by the processing unit 1503. The processes for embodiments described herein can be performed by the processing unit 1503 using computer usable program code, which can be located in a memory such as, for example, main memory 1504, ROM 1510, or in one or more peripheral devices.

A bus system 1516 can be comprised of one or more busses. The bus system 1516 can be implemented using any type of communication fabric or architecture that can provide for a transfer of data between different components or devices attached to the fabric or architecture. A communication unit such as the modem 1509 or the network adapter 1506 can include one or more devices that can be used to transmit and receive data.

Those of ordinary skill in the art will appreciate that the hardware depicted in FIG. 8 may vary depending on the implementation. Other internal hardware or peripheral devices, such as flash memory, equivalent non-volatile memory, or optical disk drives may be used in addition to or in place of the hardware depicted. Moreover, the data processing system 1500 can take the form of any of a number of different data processing systems, including but not limited to, client computing devices, server computing devices, tablet computers, laptop computers, telephone or other communication devices, personal digital assistants, and the like. Essentially, data processing system 1500 can be any known or later developed data processing system without architectural limitation.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “patient” and “subject” are interchangeable and refer to any living organism which contains neural tissue. As such, the terms “patient” and “subject” may include, but are not limited to, any non-human mammal, primate or human. A subject can be a mammal such as a primate, for example, a human. The term “subject” includes domesticated animals (e.g., cats, dogs, etc.); livestock (e.g., cattle, horses, swine, sheep, goats, etc.), and laboratory animals (e.g., mice, rabbits, rats, gerbils, guinea pigs, possums, etc.). A patient or subject may be an adult, child or infant.

The term “tissue” refers to any aggregation of similarly specialized cells which are united in the performance of a particular function.

The term “disorder” is used in this disclosure to mean, and is used interchangeably with, the terms “disease,” “condition,” or “illness,” unless otherwise indicated.

The term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

What is claimed is:

1. A system for detecting a neurological state of a brain of a patient, the system comprising:
one or more recording arrays configured to be minimally invasively inserted to a target recording site of the brain, each recording array comprising a plurality of recording electrodes having a spacing of less than about 1 mm therebetween, each recording electrode having a diameter of less than about 1 mm and configured to record electrical signals from the target recording site;
a database storing electrophysiological data therein, the electrophysiological data relating to one or more known electrophysiological signatures associated with a plurality of potential neurological states;
a processor in electrical communication with each of the one or more recording arrays and the database; and
a non-transitory, computer-readable medium storing instructions that, when executed, cause the processor to:
receive, via the one or more recording arrays, one or more recorded signals from the target recording site,
access the electrophysiological data from the database,
identify, based on the electrophysiological data, one or more electrophysiological signatures in the one or more recorded signals from amongst the one or more known electrophysiological signatures, and
determine, based on the one or more identified electrophysiological signatures, the neurological state of the brain from amongst the plurality of potential neurological states.

2. The system of claim 1, further comprising at least one stimulation array configured to be inserted to a target stimulation site of the brain, each stimulation array comprising at least one stimulation electrode configured to deliver electrical pulses to the target stimulation site,
wherein the instructions, when executed, further cause the processor to:
deliver, via the at least one stimulation array, a set of electrical pulses to the target stimulation site based on the determined neurological state, wherein the set of electrical pulses is configured to electrically stimulate the brain.

3. The system of claim 1, wherein each of the one or more recording arrays is a thin-film electrode array.

4. The system of claim 1, wherein each of the one or more recording arrays is a two-dimensional electrode array.

5. The system of claim 1, wherein each of the one or more recording arrays has a spatial density of at least about 650 electrodes/cm².

6. The system of claim 1, wherein each of the one or more recording arrays comprises at least about 1000 electrodes.

7. The system of claim 6, wherein each of the one or more recording arrays comprises about 1024 electrodes.

8. The system of claim 1, wherein each electrophysiological signature comprises one or more characteristics detectable in the one or more recorded signals.

9. The system of claim 8, wherein the one or more characteristics are selected from the group consisting of amplitude, wavelength, frequency, phase, and spatial or anatomic location of the one or more recorded signals.

10. The system of claim 1, wherein the neurological state of the brain is determined either continuously or iteratively with a recorded signal selected at intervals of about 100 ms or less.

11. A computer-implemented method for detecting a neurological state of a brain of a patient, the method comprising:
receiving one or more recorded signals from a target recording site via one or more recording arrays positioned at the target recording site;
accessing electrophysiological data from a database, the electrophysiological data relating to one or more known electrophysiological signatures associated with a plurality of potential neurological states;
identifying, based on the electrophysiological data, one or more electrophysiological signatures in the one or more recorded signals from amongst the one or more known electrophysiological signatures; and
determining, based on the one or more identified electrophysiological signatures, the neurological state of the brain from amongst the plurality of potential neurological states.

12. The method of claim 11, further comprising delivering, via at least one stimulation array, a set of electrical pulses to the target stimulation site based on the determined neurological state, each stimulation array comprising at least one stimulation electrode, wherein the set of electrical pulses is configured to electrically stimulate the brain.

13. The method of claim 11, wherein each of the one or more recording arrays is a thin-film electrode array.

14. The method of claim 11, wherein each of the one or more recording arrays is a two-dimensional electrode array.

15. The method of claim 11, wherein each of the one or more recording arrays has a spatial density of at least about 650 electrodes/cm².

16. The method of claim 15, wherein each recording array comprises a plurality of recording electrodes having a spacing of less than about 1 mm therebetween, wherein each recording electrode has a diameter of less than about 1 mm.

17. The method of claim 11, wherein each of the one or more recording arrays comprises at least about 100 recording electrodes.

18. The method of claim 17, wherein each of the one or more recording arrays comprises about 1024 recording electrodes.

19. The method of claim 18, wherein each recording array comprises a plurality of recording electrodes having a spacing of less than about 0.5 mm therebetween, wherein each recording electrode has a diameter of less than about 0.1 mm.

20. The method of claim 11, wherein each electrophysiological signature comprises one or more characteristics detectable in the one or more recorded signals.

21. The method of claim 20, wherein the one or more characteristics are selected from the group consisting of amplitude, wavelength, frequency, phase, and spatial or anatomic location of the one or more recorded signals.

22. The method of claim 11, wherein the determining the neurological state comprises determining a neurological state either continuously or iteratively with a recorded signal selected at intervals of about 100 ms or less.
